SEQUENCE LISTING (2 sequences)

SEQ ID NO: 1 - DNA, 2946 bp, Artificial Squence
  Mutant colH with E451D mutation
atggttcagaatgagagtaaacgttataccgttagctacctgaaaacactgaactattatgatctggtggatctgctggtgaagacagagattgaaaacttacctgatctgttccagtacagcagcgatgccaaagagttttacggcaacaagacccgtatgagcttcatcatggacgagattggccgtcgtgcccctcagtataccgaaatcgaccataagggcatcccgaccctggttgaggttgtgcgtgccggtttctacctgggcttccacaataaagagctgaacgaaatcaataaacgtagctttaaggagcgcgtgattcctagtattctggccattcagaaaaatcctaatttcaagctgggtactgaagtgcaggacaaaatcgttagcgccacggggttactggcaggcaatgaaaccgccccgccggaggttgtgaataacttcaccccgatcttgcaggactgcatcaaaaacatcgaccgctatgccctggacgacctgaaaagtaaggcactgttcaacgtgctggccgcacctacatacgatatcacagaatacttacgcgccaccaaggagaaaccggagaataccccgtggtacggcaagattgacggcttcattaacgagctgaaaaaactggcattatacggcaaaatcaatgacaacaacagctggattatcgataatggcatttaccacattgcacctctgggtaaactgcatagcaacaataaaattggtattgagaccctgaccgaagttatgaaagtttacccgtacctgagcatgcagcatctgcaaagcgccgatcaaatcaaacgccactacgatagcaaggatgccgaaggcaacaagatccctctggataaatttaaaaaagaaggtaaagaaaaatattgtccgaaaacctatacatttgatgatggcaaagttattattaaggcaggcgcacgcgtggaagaggagaaagtgaaacgcctgtattgggccagcaaagaggtgaacagtcagttcttccgcgtttatggcatcgacaagccgctggaagaaggcaacccggatgacattctgacgatggtgatctataacagcccggaggagtacaagctgaatagtgtgttatatggttatgataccaataatggtggtatgtatatcgagccggaaggcaccttcttcacctatgagcgtgaagcccaagagagtacctataccctggaggagctgttccgtcacgagtatacacactacctgcaaggccgctatgcagttccgggtcaatggggccgcaccaaactgtatgacaacgaccgtctgacctggtatgaggagggcggtgcagacttatttgccggtagtacccgtaccagcggtattctgccgcgtaagagcatcgttagcaacattcataataccacacgtaacaatcgttacaaactgagtgataccgttcacagcaaatacggcgcaagtttcgaattttataattacgcatgcatgtttatggactacatgtataataaagatatgggcatcctgaataaactgaatgatctggccaagaataatgatgttgacggttacgataactacatccgcgatctgagcagcaattacgccctgaatgataagtatcaggaccacatgcaggagcgcatcgacaattacgagaacttaaccgtgccgttcgttgccgacgactacctggttcgtcatgcctataagaatccgaatgaaatctacagtgaaattagcgaggttgcaaagctgaaagacgccaagagcgaggtgaaaaaaagtcagtatttcagtaccttcaccttacgcggcagttacacggggggcgccagcaagggtaagctggaagaccagaaagccatgaataaatttatcgatgatagcttaaaaaaattagatacctatagctggagtggctacaaaaccctgaccgcatacttcaccaactataaggtggatagcagtaatcgcgtgacctatgacgtggtgtttcacggctacctgccgaatgagggtgatagcaagaacagcttaccgtacggtaagatcaacggcacctacaagggcaccgaaaaggagaagattaagttcagcagtgaaggcagcttcgaccctgacggcaaaattgtgagttacgagtgggacttcggcgatggcaacaagagcaacgaggagaacccggaacacagttacgacaaggtgggcacctacacagtgaaactgaaagtgaccgatgacaaaggcgaaagcagcgttagcaccacaaccgcagagatcaaagacttaagcgagaataaactgccggtgatttacatgcacgtgccgaaaagtggcgccctgaaccagaaagtggtgttttatggcaaaggtacatacgacccggatggcagcatcgccggttatcagtgggattttggcgacggcagtgatttcagcagcgagcagaacccgagtcatgtttacaccaagaagggcgaatataccgtgaccctgcgtgtgatggacagcagcggccagatgagtgaaaaaaccatgaaaatcaaaattaccgacccggtgtacccgattggcaccgagaaagaaccgaacaacagcaaggagaccgccagcggccctatcgttcctggtattcctgttagcggcaccattgagaacacaagcgatcaggactatttctattttgatgtgatcaccccgggcgaagtgaagattgacattaacaaactgggttatggtggcgccacctgggtggtgtacgatgagaacaacaatgccgtgagttacgcaaccgacgatggccagaatctgagcggcaaattcaaagccgacaagccgggtcgctattacattcatctgtatatgttcaacggcagctacatgccgtatcgtattaacattgaaggtagcgtgggtcgc

SEQ ID NO: 2 - protein, 982 aa, Artifical Sequence
  mutant ColH with E451D mutation
MVQNESKRYTVSYLKTLNYYDLVDLLVKTEIENLPDLFQYSSDAKEFYGNKTRMSFIMDEIGRRAPQYTEIDHKGIPTLVEVVRAGFYLGFHNKELNEINKRSFKERVIPSILAIQKNPNFKLGTEVQDKIVSATGLLAGNETAPPEVVNNFTPILQDCIKNIDRYALDDLKSKALFNVLAAPTYDITEYLRATKEKPENTPWYGKIDGFINELKKLALYGKINDNNSWIIDNGIYHIAPLGKLHSNNKIGIETLTEVMKVYPYLSMQHLQSADQIKRHYDSKDAEGNKIPLDKFKKEGKEKYCPKTYTFDDGKVIIKAGARVEEEKVKRLYWASKEVNSQFFRVYGIDKPLEEGNPDDILTMVIYNSPEEYKLNSVLYGYDTNNGGMYIEPEGTFFTYEREAQESTYTLEELFRHEYTHYLQGRYAVPGQWGRTKLYDNDRLTWYEEGGADLFAGSTRTSGILPRKSIVSNIHNTTRNNRYKLSDTVHSKYGASFEFYNYACMFMDYMYNKDMGILNKLNDLAKNNDVDGYDNYIRDLSSNYALNDKYQDHMQERIDNYENLTVPFVADDYLVRHAYKNPNEIYSEISEVAKLKDAKSEVKKSQYFSTFTLRGSYTGGASKGKLEDQKAMNKFIDDSLKKLDTYSWSGYKTLTAYFTNYKVDSSNRVTYDVVFHGYLPNEGDSKNSLPYGKINGTYKGTEKEKIKFSSEGSFDPDGKIVSYEWDFGDGNKSNEENPEHSYDKVGTYTVKLKVTDDKGESSVSTTTAEIKDLSENKLPVIYMHVPKSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGSVGR

(19) United States
(12) Patent Application Publication
(10) Pub. No.: US 2021/0393501 A1
(43) Pub. Date: Dec. 23, 2021

(54) PREPARATION METHOD AND APPLICATION OF RECOMBINANT MUTANT COLLAGENASE

(71) Applicant: Rejuven Dermaceutical Co., Ltd., Hangzhou (CN)

(72) Inventors: Yong CANG, Hangzhou (CN); Zhen ZHANG, Hangzhou (CN); Gang DU, Hangzhou (CN); Fuding CHEN, Hangzhou (CN)

(73) Assignee: Rejuven Dermaceutical Co., Ltd., Hangzhou (CN)

(21) Appl. No.: 17/056,742

(22) PCT Filed: Mar. 12, 2019
(86) PCT No.: PCT/CN2019/077766

§ 371 Date: Nov. 18, 2020

(30) Foreign Application Priority Data
Jul. 30, 2018 (CN) 201810851432.1

(51) Int. Cl. A61K 8/66; A23L 33/195; C12N 9/64; A61Q 19/06; A61K 38/48

(52) U.S. Cl. CPC A61K 8/66; A23L 33/195; C12Y 304/24003; A61Q 19/06; A61K 38/4886; C12N 9/6491

(57) ABSTRACT

Provided are purification methods and uses of a recombinant mutant collagenase, and methods for preparing high-purity mutant ColH and the purified enzyme product. The method for preparing high-purity mutant ColH includes expressing recombinant mutant collagenase protein with single mutation of E451D in ColH by using specific host strain E. coli BL21 (DE3), and improving yield of the target protein after induction by low-temperature fermentation. The purification includes five steps: Capto Phenyl HS hydrophobic interaction chromatography; Capto Q anion exchange chromatography; Capto Octyl hydrophobic interaction chromatography; Phenyl HP hydrophobic interaction chromatography and Source 15Q anion exchange chromatography. The target protein obtained has purity of over 98%.

### REFERENCE TO SEQUENCE LISTING

This application includes a Sequence Listing which has been submitted electronically as a text file and is hereby incorporated by reference. The name of the text file is “SubstituteSequenceListing.txt”, which was created on Jul. 13, 2021. The size of the text file is 12,240 bytes.

### TECHNICAL FIELD

The present invention belongs to the pharmaceutical field of biological products, and relates to the purification method of recombinant mutant collagenase and its application.

### BACKGROUND

Collagenase is widely used in medical health, industrial production and scientific research, such as debridement, treatment of lumbar intervertebral disc hemiation and treatment of rare diseases such as Dupuytren's Contracture and Peroni's Disease. It is expected to help develop new drugs for dissolving lipid, reducing scar and skin micro-plastic, and be used in food softening in industrial production, cell separation, and processing of archaeological samples in scientific research, etc.

With the improvement of people's living standard, obesity is becoming more and more common, and it is a big trouble for people who love beauty. At present there are many products in the weight loss market. Liposuction, a widely used method of reducing fat, is a physical method with the aid of instruments. It will cause certain damage to the body and other tissues in the liposuction site are easily damaged. It is also prone to side effects such as infection, bruising, hematoma, deep venous thrombosis and so on. At present there are laser-assisted lipid-dissolving, ultrasound-assisted lipid-dissolving and injection-assisted lipid-dissolving methods, as well as non-invasive lipid-dissolving ones including frozen lipid-dissolving, radio frequency, ultrasound and etc.

Local subcutaneous adipose mass, such as double chin, is an indication resulting from fat accumulation, which is difficult to eliminate through physical exercises. In 2015 the Food and Drug Administration (FDA) approved the world's first “double chin” lipolytic injection Kybella (ATX-101) for treating moderate to severe“double chin” adults. This is the first and only non-surgical treatment product to eliminate excess submental fat (double chin). Kybella is a synthetic deoxycholic acid, which mainly acts on the cell membrane and causes cell rupture, thus achieving lipolysis. Because of the action mechanism of deoxycholic acid Kybella has no specificity. Besides adipocytes Kybella can also act on other cells; therefore, Kybella has great side effects. It is easy to cause mandibular marginal nerve injury, dysphagia, hematoma or stasis at injection sites and so on.

With the discovery and application of collagenase, its special mechanism of action enables it to be applied in the field of lipolysis. Currently, commercial collagenase (including collagenase from Clostridium histolyticum) is extracted directly from biological samples. Because many isoenzymes exist in organisms, these commercial varieties are often composed of 5-6 kinds of collagenase. ColH and ColG with similar molecular weights and isoelectric points (PIs) are difficult to be separated and purified. Therefore, highly purified collagenase from Clostridium histolyticum is still a mixture comprising ColG and ColH. Xiaflex, which came into the market in 2010, is such a mixture of ColH and ColG

Because of the non-singularity of components, there are many limitations in applications of Xiaflex, such as bleeding easily caused by injection in animals and side effects which are not easy to control. Secretory expression of ColH in Clostridium perfringens was conducted by Eiji Tamai et al. (Eiji Tamai et al., High-level expression of his-tagged clostridial collagenase in Clostridium perfringen, Appl Microbiol Biotechnol (2008) 80:627-635). In order to facilitate purification, the C-terminal of ColH contained His-tag. Recombinant ColH with purity of about 90% was obtained by ammonium sulfate precipitation, zinc affinity chromatography and Mono Q anion exchange chromatography. Paulina Ducka et al. (Paulina Ducka et al., A universal strategy for high-yield production of soluble and functional clostridial collagenases in E. coli, Appl Microbiol Biotechnol (2009) 83:1055-1065) expressed ColH using E. coli and obtained recombinant ColH with purity of about 90% through nickel column affinity purification, anion exchange chromatography and molecular sieve chromatography. However, the purity and quality control of ColH obtained from these studies are difficult to meet requirements of clinical application.

Purified collagenase from commercial sources (including Clostridium histolyticum) is a mixture of 5-6 collagenase proteases. Since ColH and ColG with similar molecular weight & isoelectric point (PIs) are difficult to be separated even through strict purification, the highly purified collagenase from Clostridium histolyticum still contains a mixture of ColG and ColH. In our obese rat experiments, large amount of bleeding was induced by wild type collagenase obtained through routine purification.

CN101678088 discloses an application of a recombinant mutant collagenase in lipolysis. The sequence of the recombinant mutant collagenase contains a GST tag and has a peptide motif before ColH (Glu451Asp). The purity of the protein product is about 90% through affinity chromatography on nickel column; however it is difficult to industrialize and marketize the drug. There are two main problems: (1) the purity of protein is low. Protein purification has always been a problem affecting the industrialization of protein drugs. The sources of impurities in protein drugs include: a. process-related impurities such as host cell components and endotoxin. b. impurities produced in downstream processes such as protein solubilizers, reductants, denaturants, trace metals and purified chromatographic ligands. c. impurities in products such as precursors, misfolded proteins, product fragments and some degradation products. Impurities may cause potential health risks (carcinogenicity, allergy, antigenicity, general or special toxicity), so the purity of therapeutic protein drugs is generally required to be more than 95%. (2) The product contains GST tags, which belong to non-natural sequence. While affinity chromatography is a commonly used method for protein separation and purification. GST is one of the most commonly used affinity chromatography purification tags. Recombinant proteins with this tag can be purified by cross-linked glutathione chromatography medium, but GST on the protein must be folded properly to form a spatial structure that binds to glutathione in order to be purified by this method. Furthermore, such a large label (GST tags contain up to 220 amino acids) may affect the solubility of expressed proteins and cause formation of inclusion bodies, which will destroy the natural structure of proteins and make it difficult to carry out structural analysis. Sometimes, the problem will not be necessarily solved if the GST label is removed after purification by enzyme digestion (O U Qin, L I N Xuesong edit. Experimental Courses of Biochemistry and Molecular Biology, 2nd Edition, Peking University Medical Press, 2015.08, Page 18).

### SUMMARY

The invention relate to a composition comprising high-purity recombinant mutant ColH (Serial Number: RJV001). Wild-type collagenase has high activity, and degrades collagen more vigorously and easily causes side effects. Moreover, wild-type collagenase is a mixture of different enzymes, which is not conducive to Chemistry Manufacturing and Control (CMC). It is difficult to control the proportion of components in each batch of products, which has a certain risk for subsequent application in human body. In order to obtain industrialized ColH, inventors of the present invention reduce the catalytic activity of ColH expressed in E. coli with E451D mutation, which makes it relatively mild when acting on animal tissues. This is more conducive to the development of new drugs, which will be used for more indications. Specifically the specific activity of mutant ColH is about 10% of that of wild-type ColH. Compared with wild-type ColH, its Kₘ value does not change much, but the K_{cat} value decreases significantly. (The Kₘ value is Michaelis constant, which is used to measure the affinity between enzymes and substrates. K_{cat} is also called transformation number, which is calculated by dividing Vₘₐₓ by enzyme concentration. So it is known that K_{cat} measures the rate at which enzymes catalyze the formation of substrates under optimal conditions. K_{cat} is a constant whose unit is 1/s; it can also be understood as the number of substrates converted by a single enzyme molecule in one second, or the time required for a single enzyme molecule to convert one substrate molecule.) Therefore, mutant ColH has a milder catalytic effect than wild-type ColH and can shear collagen slowly. In addition, after obtaining high purity (more than 98%) of mutant collagenase, its stability is investigated and it is found that mutant collagenase has better stability.

The composition of embodiments of the present invention may further comprise a pharmaceutically acceptable carrier. Pharmaceutically acceptable carriers include those inert to mutant ColH, such as a group comprising saline, dextran and hydroxyethyl starch aqueous solutions. In a preferred embodiment the pharmaceutically acceptable carrier is a buffer with neutral pH. In addition, fibrin glue can be used as the pharmaceutically acceptable carrier, including fibrin or fibrin precursor such as fibrinogen plus thrombin.

On the other hand, embodiments of the present invention develop a process for producing high-purity Clostridium histolyticum collagenase by E. coli. By investigating fermentation conditions and optimizing culture medium, most of the target proteins expressed in E. coli are soluble and the fermentation period is shorter with higher yield of collagenase and stable catalytic activity. After being harvested, the cells are homogenized by high-pressure homogenate, filtered and clarified by hollow fiber column and purified by five-step column chromatography. Finally collagenase with purity of more than 98% is obtained, for which adding protective agents such as human serum albumin is not needed, and it is stable at 2° C.-8° C. or at −70° C.

Specifically, the recombinant mutant collagenase in embodiments of the present invention is expressed in E. coli. After fermentation and homogenization by high-pressure homogenate a qualified product is obtained by five-step column chromatography of the supernatant. The five steps of purification (AKTA purification system) are as follows:

Step 1: Capto Phenyl HS hydrophobic interaction chromatography: equilibrating the Capto Phenyl HS hydrophobic chromatography column, precipitating with ammonium sulfate and resuspending, loading the supernatant onto the Capto Phenyl HS hydrophobic chromatography column, washing and eluting, collecting an elution peak and obtaining first-collected solution;

Step 2: Capto Q anion exchange chromatography: loading the first-collected solution of Step 1 onto the Capto Q anion exchange chromatography column, washing and eluting, collecting a main elution peak and obtaining solution collected for the second time;

Step 3: Capto Octyl hydrophobic interaction chromatography: equilibrating the Capto Octyl hydrophobic interaction chromatography column, loading the solution from Step 2 onto the Capto Octyl hydrophobic interaction chromatography column, washing and eluting, collecting a main elution peak and obtaining solution collected for the third time;

Step 4: Phenyl HP hydrophobic interaction chromatography: loading the solution from Step 3 after high-concentration salt treatment onto the Phenyl HP hydrophobic interaction chromatography column, washing and eluting, collecting a main elution peak and obtaining solution collected for the fourth time;

Step 5: Source 15Q anion exchange chromatography: equilibrating the Source 15Q anion exchange chromatography column, loading the solution from Step 4 onto the Source 15Q anion exchange chromatography column, washing and eluting, collecting a main elution peak and obtaining solution collected for the fifth time;

Displacing the buffer of the fifth collected solution of the step 5 by ultrafiltration, and obtain a final product by concentrating, filtering, sterilizing and freeze-drying.

Details for the five steps of purification (AKTA purification system) are as follows:

Step 1: Capto Phenyl HS Hydrophobic Interaction Chromatography;

Clean Capto Phenyl HS hydrophobic interaction chromatography system to remove pyrogen. Equilibrate the column with mobile phase A, load samples and wash the column with mobile phase A. The elution peaks are collected by gradient or isocratic elution with mobile phase B and first-collected solution is obtained. Wherein the mobile phase A is 30-80 mM Tris, 1-2 M NaCl with pH 7.0-9.0, and is preferably 30, 50 and 80 mM Tris with pH 7.0, 8.0 and 9.0. The mobile phase B is 30-80 mM Tris-HCl with pH 7.0-9.0 and is preferably 30, 50, 80 mM Tris with pH 7.0, 8.0 and 9.0.

Step 2: Capto Q Anion Exchange Chromatography;

Equilibrate the column with mobile phase A, load the solution collected in Step 1, and wash the column with mobile phase A. The elution peaks are collected by gradient or isocratic elution with mobile phase B and solution collected for the second time is obtained. Wherein the mobile phase A is 30-80 mM Tris with pH 7.0-9.0 and is preferably 30, 50 and 80 mM Tris with pH 7.0, 8.0 and 9.0. The mobile phase B is 30-80 mM Tris-HCl, 0.1-1M NaCl with pH 7.0-9.0, and is preferably 30, 50, 80 mM Tris. 0.1, 0.5 and 1M NaCl with pH 7.0, 8.0 and 9.0.

Step 3: Capto Octyl Hydrophobic Interaction Chromatography;

Equilibrate the Capto Octyl hydrophobic interaction chromatography column with mobile phase A and load the solution collected in Step 2. The main elution peaks are collected by gradient or isocratic elution with mobile phase B and solution collected for the third time is obtained. Wherein the mobile phase A is 30-80 mM Tris, 1-2M NaCl with pH 7.0-9.0, and is preferably 30, 50 and 80 mM Tris with pH 7.0, 8.0 and 9.0. The mobile phase B is 30-80 mM Tris-HCl with pH 7.0-9.0, and is preferably 30, 50, 80 mM Tris with pH 7.0, 8.0 and 9.0.

Step 4: Phenyl HP Hydrophobic Interaction Chromatography;

Equilibrate the Phenyl HP hydrophobic interaction chromatography column with mobile phase A. and load the solution collected in Step 3 after high-concentration salt treatment. The main elution peaks are collected by gradient or isocratic elution with mobile phase B and solution collected for the fourth time is obtained. Wherein the mobile phase A is 30-80 mM Tris, 1-2M NaCl with pH 7.0-9.0, and is preferably 30, 50 and 80 mM Tris with pH 7.0, 8.0 and 9.0. The mobile phase B is 30-80 mM Tris-HCl with pH 7.0-9.0, and is preferably 30, 50.80 mM Tris with pH 7.0, 8.0 and 9.0.

Step 5: Source 15Q Anion Exchange Chromatography;

Equilibrate the Source 15Q anion exchange chromatography column with mobile phase A and load the solution collected in Step 4. The main elution peaks are collected by gradient or isocratic elution with mobile phase B and solution collected for the fifth time is obtained. Wherein the mobile phase A is 30-80 mM Tris with pH 7.0-9.0, and is preferably 30, 50 and 80 mM Tris with pH 7.0, 8.0 and 9.0. The mobile phase B is 30-80 mM Tris-HCl, 0.1-1M NaCl with pH 7.0-9.0, and is preferably 30, 50, 80 mM Tris & 0.1, 0.5, 1M NaCl with pH 7.0, 8.0 and 9.0.

After fermentation with a 65-L fermentor, the fresh weight of bacteria was 65-80 g/L and the enzyme activity was 25-35 U/g. After clarification and 5-step chromatography purification, the purity of collagenase was more than 98%. The specific activity of the obtained collagenase was 1.1-1.4 U/mg and the total purification yield was 10-20%. When it was stored at −80° C. or 2° C.-8° C., there was no obvious polymer generated or activity loss. The mutant collagenase produced by this process has high purity and good stability. Compared with previous collagenase products, it has obvious advantages and specific activity of the enzyme is significantly increased.

Embodiments of the present invention also relate to a method for reducing adipose tissue at a designated position in the body, including introduction of an effective amount of highly purified mutant collagenase into the tissue.

According to embodiments of the present invention, the high-purity mutant collagenase can be used as a fat-decomposing cream accompanied by transdermal technology or as an epidermal cream to replace liposuction. In other words, embodiments of the present invention provide a new method for reducing excessive amount of unsightly and/or redundant subcutaneous adipose tissue, which is a non-invasive method such as injection or epidermal cream.

High-purity mutant collagenase can be developed as a drug because it is a single substance with higher purity and less impurities, which makes it easy to carry out Chemistry Manufacturing and Control (CMC). When high-purity mutant collagenase is introduced into subcutaneous adipose tissue of living animals, the adipose tissue is decomposed and reduced. This method is mild and accurate; it will not cause human trauma, nor will it cause infection.

In embodiments of the present invention, the final products were diluted with saline to 0.015 mg/point, 0.05 mg/point, 0.15 mg/point & 0.25 mg/point, and injected into fat layers on back sides of mini pigs. It is found that the fat layer of the injection site was significantly reduced through ultrasound examination and anatomic examination, which showed that the purified recombinant mutant collagenase had significant effects on lipid elimination. An additional application of the present invention is scar reduction, whether found on the skin surface or not. High-purity mutant collagenase can digest collagen in protruding scar tissue, thereby reducing the height and appearance of scars.

The present invention can also be used to treat lipoma and other adipose tissues, which can be applied in human and animal bodies, in wildlife, in human homes, or in zoos.

Other features and advantages of the present invention will be described in detail in subsequent embodiments.

### BRIEF DESCRIPTION OF THE DRAWINGS

FIG. 1 depicts a nucleotide sequence of mutant ColH with E451D single point mutation.

FIG. 2 depicts a protein sequence of mutant ColH with E451D single point mutation.

FIG. 3 depicts screening of host bacteria. Compared with BL21 (DE3) playS, BL21 (DE3) can express more target proteins; while Transetta cannot express enough target proteins, which cannot meet the requirements of further experiments.

FIG. 4 depicts screening of the temperature of expression. Compared with 37° C., lower temperature of 28.5° C. can significantly improve protein expression.

FIG. 5 depicts the purity of protein purified through four-step purification including Capto Q, Capto Octyl, Phenyl HP and Source 15Q; Capto Phenyl HS hydrophobic interaction chromatography is not applied. SDS-PAGE & grayscale analysis results show that purity of the obtained protein is only 94.6%, which cannot meet production requirements.

FIG. 6 depicts comparison between Capto Q anion exchange chromatography and Capto DEAE anion exchange chromatography. The results show that the separation degree of Capto DEAE is poor and suitable target protein cannot be obtained. On the contrary, the separation degree of Capto Q is good and is suitable for the target protein separation of embodiments of the present invention.

FIG. 7 depicts the purity results of five-step purification by SDS-PAGE.

FIG. 8 depicts the grayscale analysis results of five-step purification by SDS-PAGE. After five steps of purification, the purity of protein products reached 99.5%.

FIG. 9 depicts the results of five-step purification by CE-SDS; the purity of protein products reached 98.740%.

FIG. 10 depicts the results of five-step purification by SEC; the purity of protein products reached 98.8%.

FIG. 11 depicts the analysis of endotoxin in each step of the five-step purification.

FIG. 12 depicts the in vitro specific activity and Kₘ value of mutant collagenase (RJV001) with high purity (>98%) through five steps of purification and those of mutant collagenase (rColH (FM)) with low purity (˜90%) through one-step purification by nickel column. The specific activity of RJV001 significantly increased and Kₘ significantly decreased with statistical difference. The higher the specific activity is, the higher the enzyme activity per unit mass will be. The smaller the Kₘ is, the stronger the ability of the enzyme binding the substrates will be. (*p<0.05; **p<0.01).

FIG. 13 depicts the pH investigation on the stability of the drug products (one month).

FIG. 14 depicts the calcium ion investigation on the stability of the drug products (three months).

FIG. 15 depicts the stability investigation of the drug substance after repeated freezing & thawing.

FIG. 16 depicts the stability investigation of the drug substance at −70° C.

FIG. 17 depicts the stability investigation of the drug substance at −20° C.

FIG. 18 depicts the in vivo ultrasound results in lipolysis pre-study of Bama miniature pig (partly).

FIG. 19 depicts the epidermic observation results in lipolysis pre-study of Bama miniature pig (partly).

FIG. 20 depicts the anatomy results in lipolysis pre-study of Bama miniature pig (partly).

FIG. 21 depicts the in vivo ultrasound statistics results in lipolysis pre-study of Bama miniature pig (partly).

FIG. 22 depicts the dosage regimen in lipolysis study of Bama miniature pig.

FIG. 23 depicts the treatment layout in lipolysis study of Bama miniature pig.

FIG. 24 depicts the mean relative thickness of each area in lipolysis study of Bama miniature pig.

FIG. 25 depicts the relative thickness of each area in lipolysis study of Bama miniature pig.

FIG. 26 depicts the relative thickness in lipolysis study of Bama miniature pig.

FIG. 27 depicts the histopathological statistics results in lipolysis study of Bama miniature pig.

FIG. 28 depicts the histopathological results in lipolysis study of Bama miniature pig.

### DETAILED DESCRIPTION

### Embodiment 1, Construction of Recombinant Mutant Collagenase Strain

### Experimental Methods

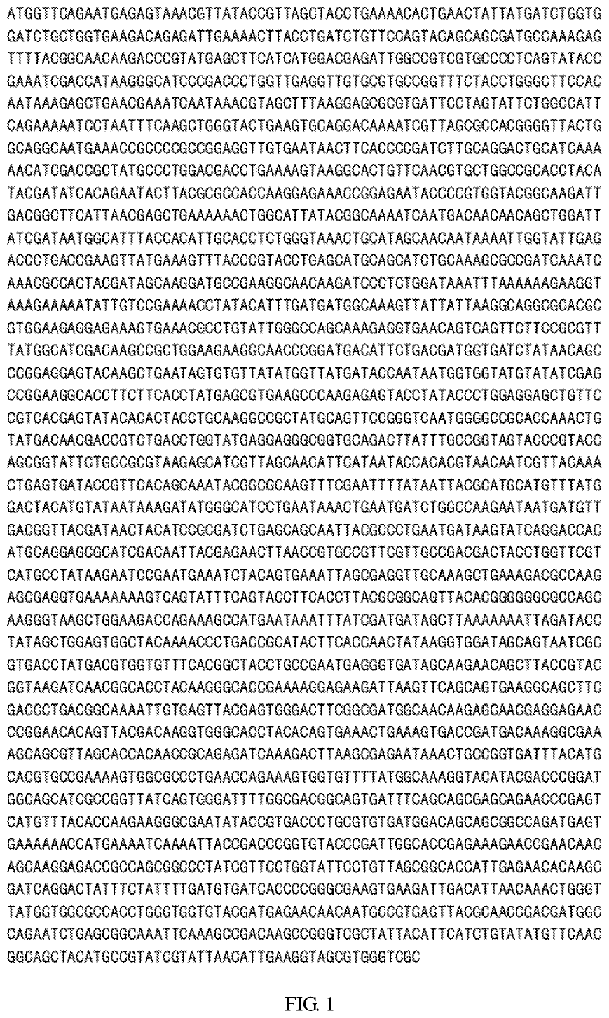

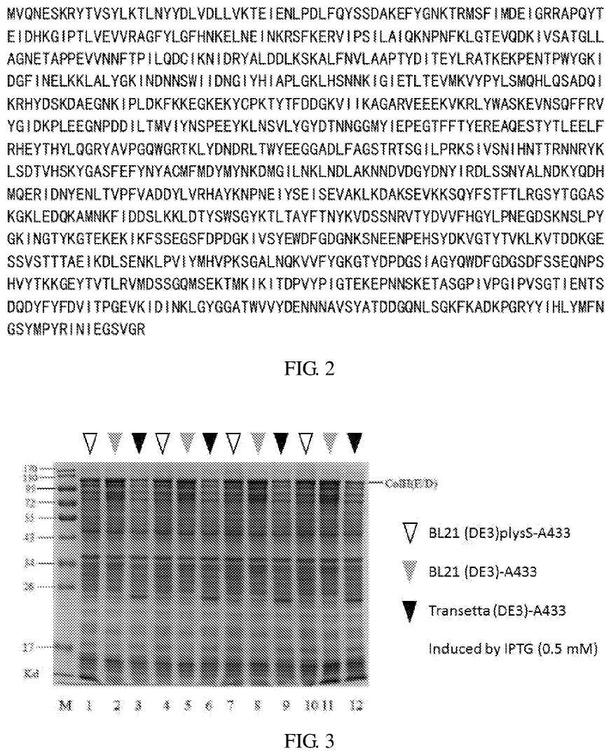

FIG. 1 depicts a nucleotide sequence of mutant colH with E451D single point mutation. FIG. 2 depicts a protein sequence of mutant ColH with E451D single point mutation. The plasmid containing synthesized mutant colH and pET-30a (+) were digested by NdeI/XhoI digestion and detected by electrophoresis; the target fragment and vector fragment were extracted. The two fragments were linked by T4 DNA ligase, and the 10 μL ligatures were transformed into 100 μL competent cells. Colonies were selected by spreading plate, and the target strain was chosen by sequencing.

Effects of different host stains on protein expression were investigated. The results in FIG. 3 show that compared with BL21 (DE3) playS, BL21 (DE3) can express more target proteins; while Transetta cannot express enough target proteins, which will not meet the requirements of further experiments.

### Embodiment 2, Fermentation of Recombinant Mutant Collagenase Strain

Instruments and Materials

BIOFLO 610 65.0-L fermenter was purchased from Eppendorf Company; high-speed freezing centrifuge was purchased from Thermo Company; working seed bank was established, tryptone and yeast extract were purchased from OXID Company; various reagents were purchased from Sinopharm Chemical Reagent Company.

Experimental Methods

The seeds were cultured in a shaking flask overnight and then were inoculated into the seeding tank under suitable conditions. After cultivation, the amplified seeds were transferred into fermentor. The medium comprises peptone 13.5051 g/L, yeast powder 7 g/L and magnesium sulfate 0.4 g/L. Cultivate it at 37° C. for 4 h. Then reduce the temperature and add IPTG at a final concentration of 0.5 mM and induction for 7-8 hours. Fed batch cultivation is conducted in this fermentation process. The dissolved oxygen and pH were monitored; OD600 and original enzyme activity were tested. After fermentation, the cells were collected by centrifugation.

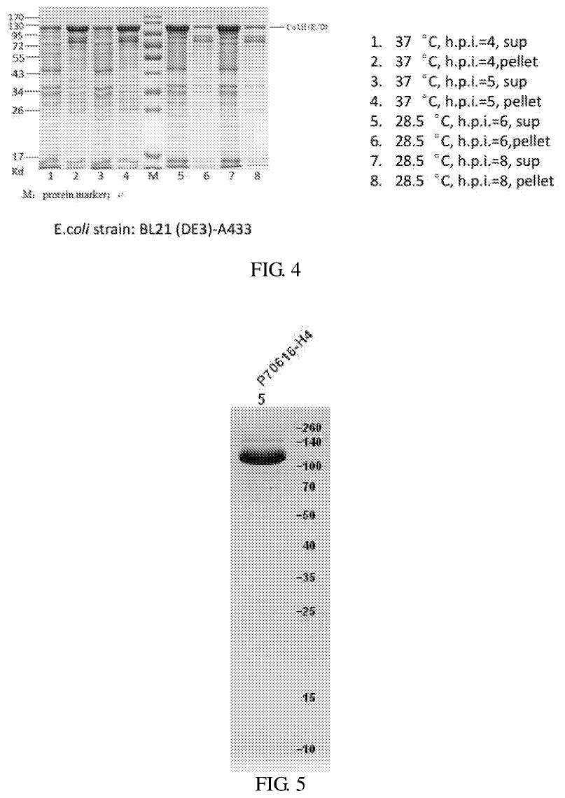

Expression temperature is an important factor affecting protein solubility; therefore it was screened during expression. It was found that the soluble protein yield could be increased by reducing fermentation temperature from 37° C. to a lower, one such as 32° C., 31.5° C., 30° C., 29.5° C., 29° C., 28.5° C., 28° C. 27.5° C. and 27° C. The results of FIG. 4 show that expression at a lower temperature around 28° C. can significantly increase the yield of soluble target protein.

### Embodiment 3, Purification Method of Recombinant Mutant Collagenase

Instruments and Materials

Packing materials such as Capto Phenyl HS. Capto Q, Capto Octyl & Phenyl HP were purchased from GE Company. Akta Purifier Chromatography System was also purchased from GE and Hollow Fiber Column Ultrafiltration System was purchased from Pall.

Experimental Methods

1) Cells Harvesting and Clarification

After fermentation the cells were collected by centrifugation, and after enlargement they could be collected by membrane treatment. Fresh bacteria can be preserved by freezing, or be crushed and used directly in the next step. The cells were suspended in Tris buffer with 10-20% suspension concentration and were homogenized under pressure of 600-700 bar by high pressure homogenizer. The cells were homogenized three times and temperature was controlled at 2-8° C. during homogenization.

The lysate was filtered by 0.65 μm hollow fiber membrane column (at a certain pump pressure). Cell fragments and soluble components were separated, clarified solution was obtained. Calculate the clarity & yield.

2) Capto Phenyl HS Hydrophobic Interaction Chromatography

Clean Capto Phenyl HS hydrophobic interaction chromatography system to remove pyrogen. Pump B was filled with solution C and Pump A was connected to solution D to equilibrate the Capto Phenyl HS hydrophobic interaction chromatography column. After equilibrating it to baseline for 2-5 CV, Pump A was transferred to sample solution, which was loaded at a flow rate of 50-90 cm/h. After that Pump A was connected with Solution D and was washed with it to baseline for 2-5 CV. Use 55% B to remove impurities to baseline for 2-5 CV. The target protein was eluted with 100% B; elution peaks were collected and recorded as solution collected in Capto Phenyl HS. Wash the Capto Phenyl HS hydrophobic interaction chromatography column.

The results in FIG. 5 indicate purity of protein after four-step purification including Capto Q, Capto Octyl, Phenyl HP and Source 15Q, wherein Capto Phenyl HS hydrophobic interaction chromatography is not applied. Purity of the finally-obtained protein is only 94.6%, which cannot meet the production requirements. Therefore in order to further increase hydrophobicity, Capto Phenyl HS hydrophobic interaction chromatography was added. The results showed that purity of the finally-obtained protein was increased to 99.5% (FIG. 8).

3) Capto Q Anion Exchange Chromatography

Refill Pump B with Solution B and Pump A with Solution A; equilibrate the Capto Q column with Solution A. After equilibrating it to baseline for 2-5 CV, Pump A was transferred to solution collected in Capto Phenyl HS with loading at a flow rate of 50-90 cm/h. After that Pump A was refilled with Solution A and the column was washed to baseline for 2-5 CV. Continue to wash the column with 5%. 10% and 15% B until the average value of ultraviolet absorption was only about 20 mAu; then wash it with 20%, until the ultraviolet absorption value was about 200 mAu. 30% B was used for eluting the target protein. The elution peaks were collected and recorded as solution collected in Capto Q. Elution effect was investigated and 40% B & 60% B were used for washing.

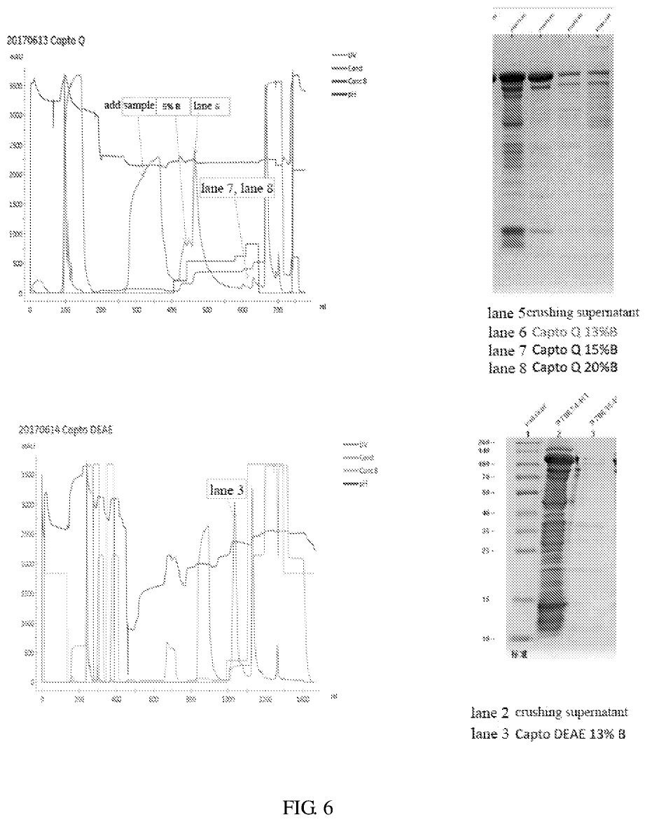

An appropriate exchange medium should be selected for anion exchange chromatography according to the target protein. In FIG. 6 strong anion exchange chromatography of Capto Q was compared with weak anion exchange chromatography of Capto DEAE. The results showed that separation degree of Capto DEAE was poor and suitable target protein could not be obtained. On the contrary the separation degree of Capto Q was good and was suitable for target protein separation of embodiments of the present invention.

4) Capto Octyl Hydrophobic Interaction Chromatography

Refill Pump B with Solution E and Pump A with Solution F; equilibrate the Capto Octyl column until baseline for 2-5 CV. Regulate conductivity of solution collected in Capto Q by diluting with Solution G, so that its conductivity was close to that of Solution F. Refill Pump A with solution collected in Capto Q and load it at 50-90 cm/h. After that refill Pump A with Solution F to wash impurities to baseline for 2-5 CV. Clean it with 10% and 15% B sequentially until no obvious UV absorption peak appeared, and then clean it with 20% B. The target protein was eluted with 37.5% B; elution peaks were collected and recorded as solution collected in Capto Octyl. Elution effect was studied subsequently by washing the column with 50% B, 60% B and 100% B.

5) Phenyl HP Hydrophobic Interaction Chromatography

Refill Pump B with Solution C and Pump A with Solution D; equilibrate the Phenyl HP column until baseline for 2-5 CV. Regulate conductivity of solution collected in Capto Octyl by diluting with Solution G, so that its conductivity was close to that of Solution D. Refill Pump A with solution collected in Capto Octyl and load it at 50-90 cm/h. Then refill Pump A with Solution D to wash impurities to baseline for 2-5 CV. After that wash it with 60-90% B to remove impurities and wash the column to baseline for 2-5 CV. Finally the target protein was eluted with 95% B, and the elution peaks were collected and recorded as solution collected in Phenyl HP. Wash the column with water.

6) Source 15Q Anion Exchange Chromatography

Refill Pump B with Solution B and Pump A with Solution A; equilibrate the Source 15Q column until baseline for 2-5 CV. Dilute the solution collected in Phenyl HP for 5 times with purified water and load it at a flow rate of 60 cm/h. Then wash Pump A with Solution A and clean it with 10% B & 12% B. The target protein was eluted with 20% B and the elution peaks were collected.

7) Ultrafiltration Concentration and Displacement Buffer

The target protein collected in Source 15Q anion exchange chromatography was displaced with a final buffer (Tris 2.2 g/L, pH 7.30*0.10), concentrated by Millipore Pellicon Ultrafiltration System. The pore size of the membrane was 10 KD.

8) Vacuum Freeze-Drying

The concentrated protein was distributed and vacuum freeze-dried.

The purification scheme of the invention is to realize the high purity preparation of recombinant mutant ColH for the first time. The product meet the industrialization quality and scale requirements after analysis.

In addition, the inventors tested the five-step purification procedure to investigate its effect on the purity of the finally-purified recombinant collagenase. The results showed that after purification including 1) Capto Phenyl HS hydrophobic chromatography, 2) Capto Q anion exchange chromatography, 3) Capto Octyl hydrophobic chromatography, 4) Phenyl HP hydrophobic chromatography & 5) Source 15Q anion exchange chromatography purity of the obtained protein was over 98% or even higher than 99%. Through the five-step purification purity of the finally-obtained recombinant collagenase can reach 98%. However if any step is omitted, such purity can hardly reach 95%. Therefore the five steps and sequence of the five-step purification will affect purity of the finally-obtained protein.

### Embodiment 4, Analysis of Recombinant Mutant Collagenase

1) SDS-PAGE

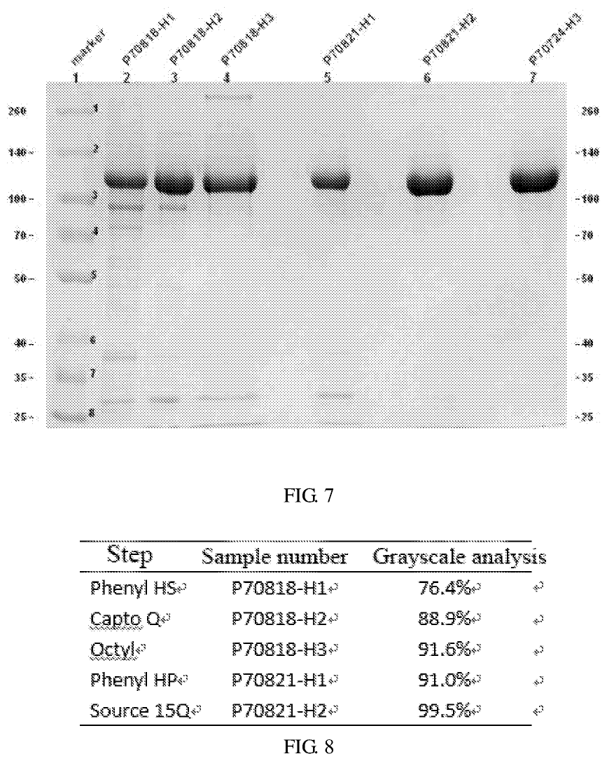

SDS-PAGE was used to detect the target proteins, which were purified by five-step purification. The results were shown in FIG. 7. The molecular weight of the target protein was consistent with that of the standard substances. FIG. 8 showed that purity of the target protein was over 99%.

2) CE-SDS

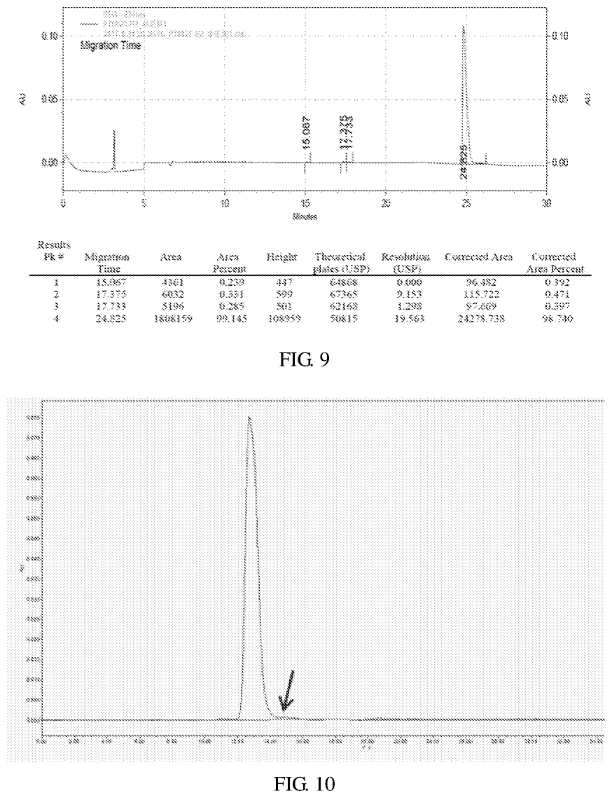

Samples were analyzed by non-reducing CE-SDS according to the method of Chinese Pharmacopoeia. The results are shown in FIG. 9.

3) Size Exclusion Chromatography (SEC-HPLC)

SEC column was from GE company. Mobile phase: 20 mM PBS, PH 7.4; detection wavelength of 280 nm. The results are shown in FIG. 10.

4) Investigation of Endotoxin

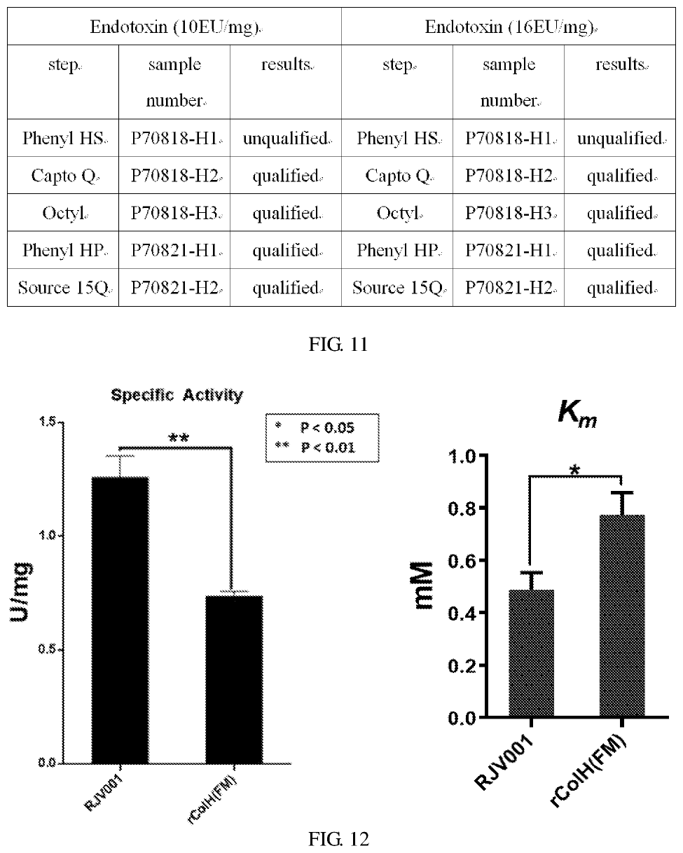

The test of endotoxin was according to the method of Chinese Pharmacopoeia. The results are shown in in FIG. 11.

5) Biochemical Activity Assay

(1) Preparations: prepare a number of 1.5-mL EP tubes & 10-mL plastic centrifuge tubes, and label them according to sample names; set the water bath temperature as 25° C.; start the ultraviolet spectrophotometer, and set the wavelength as 320 nm.

(2) Preparation of reaction system: transfer 0.1M CaCl₂ solution into a 1.5-mL EP tube with pipettor, add into it 1 mL substrate solution and mix them. Keep the mixture in water bath at 25° C.

(3) Enzymatic reaction: when the temperature of reaction system was reached to 25° C., add 50 μL samples according to the labels. Replace the blank control with 50 μL 0.1M Tris buffer. Then keep samples in the water bath again for 15 minutes exactly.

(4) Drying tube: weigh about 0.37 g anhydrous sodium sulfate, put it into a 10-mL centrifuge tube and cover it.

(5) Extraction solution: add 1 mL citric acid solution into a 10-mL centrifuge tube; then add 5 mL ethyl acetate, which was on the upper layer of citric acid solution. Close the lid.

(6) When time was up, transfer 0.5 mL reaction system immediately into the extraction solution with pipettor vortex for 20 seconds. At this time the upper layer of ethyl acetate was turbid; move 3 mL of the upperlayer into a 10-mL Drying tube and shake it immediately. Then ethyl acetate became clarified.

(7) Measure A320: first test blank control and then test each sample; the best reading of A320 should be between 0.3 and 0.9.

(8) Formula for calculating enzyme activity

enzyme activity (U/ml)=(A−A_{B})×[V_{T}×V_{E}/(ε×V×V_{R}×B×T)]×D

A=Absorption Value of Standard Substances and Samples

A_{B}=Absorption value of blank control

V_{T}=Reaction volume, 1.25 mL

V_{E}=Volume of ethyl acetate in extraction solution, 5 mL

ε=Molar Absorption Coefficient of 320 nm in Extraction Solution. 21 mL/(μmol cm)

V=Volume of the added samples or standard substances, 0.05 mL

V_{R}=The reaction volume transferred to the extraction solution, 0.5 mL

B=Optical path, 1 cm

T=Enzymatic reaction time, 15 min

D=Dilution factor

Enzyme activity of the freeze-dried product was determined and experimental data were shown in FIG. 12. Wherein rColH (FM) is a mutant collagenase purified by one-step nickel column with purity of about 90%. Its 451 site glutamic acid is mutated into aspartic acid and contains His tag. While RJV001 is a mutant collagenase purified by five-step purification in the present specification; its 451 site glutamic acid is mutated to aspartic acid and does not contain OST or His tag.

| | Purity | 451D Mutant | His-tag |
|---|---|---|---|
| rColH(FM) | −90% | Y | Y |
| RJV001 | >98% | Y | N |

FIG. 12 shows that specific activity of low-purity rColH (FM) is 0.74 U/mg, while that of high-purity RJV001 obtained by embodiments of the present invention is 1.10 U/mg. There is a significant difference between above two products (p<0.05), which proves that purity of the product is improved by methods of the present specification and specific activity is significantly improved. In addition, Kₘ of RJV001 decreased significantly, indicating that the affinity of RJV001 to substrate was significantly greater than that of rColH (FM).

6) Stability Study

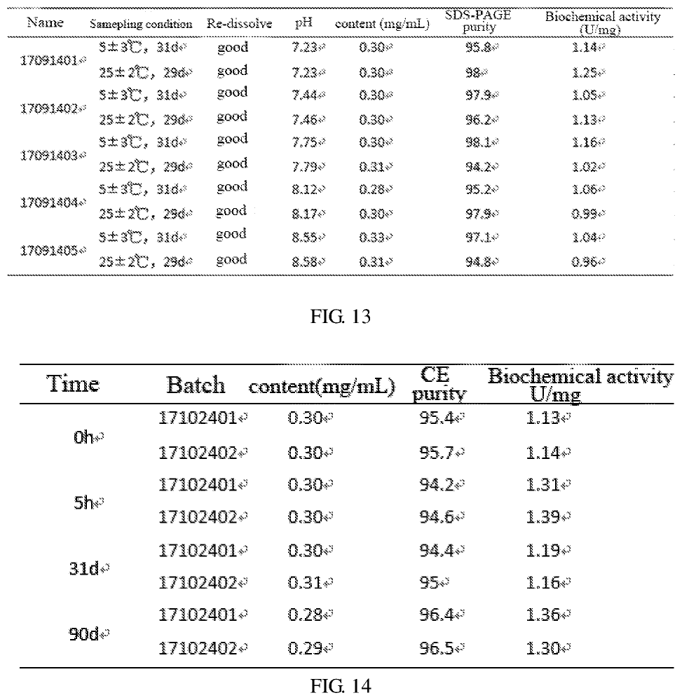

The effects of pH, calcium ion and freeze-drying time on biochemical activity of drug products, and the effects of repeated freezing & thawing, storage at different temperature (40° C., room temperature, low temperature, −70° C.) on biochemical activity of drug substance were investigated. The experimental results are shown in FIGS. 13-17.

FIG. 13 shows that, when the range of pH increased from 7.23 to 8.58, biochemical activity of RJV001 at neutral pH was well maintained at either 5° C. or 25° C.; while at weak alkaline pH the biochemical activity decreased slightly.

FIG. 14 shows that, the addition of calcium ions in two batches had no significant effects on biochemical activity of freeze-dried drug products of RJV001 in three months.

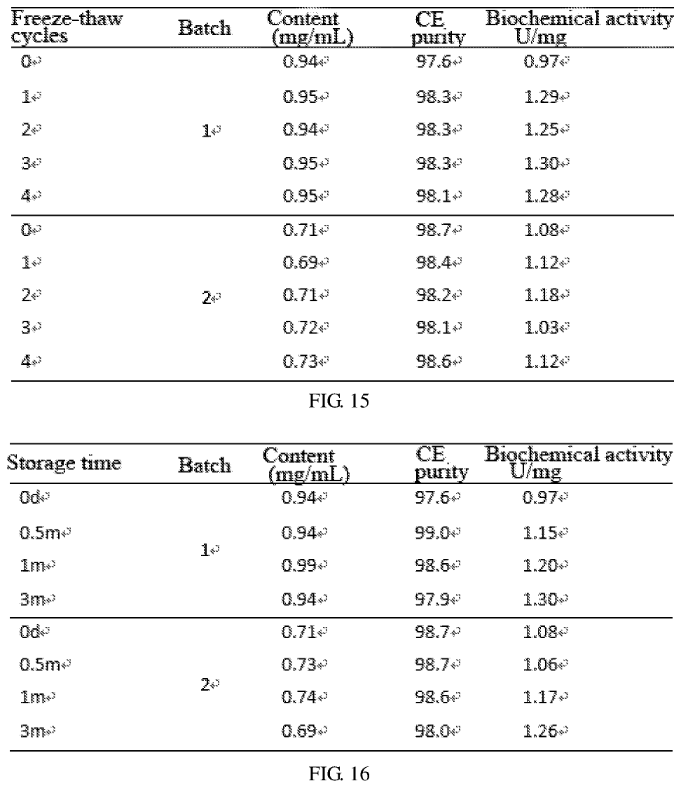

FIG. 15 shows that, four freezing-thawing cycles had no significant effects on the biochemical activity of two RJV001 batches.

FIG. 16 shows that, storage at −70° C. for three months had no effects on biochemical activity of RJV001 drug substance.

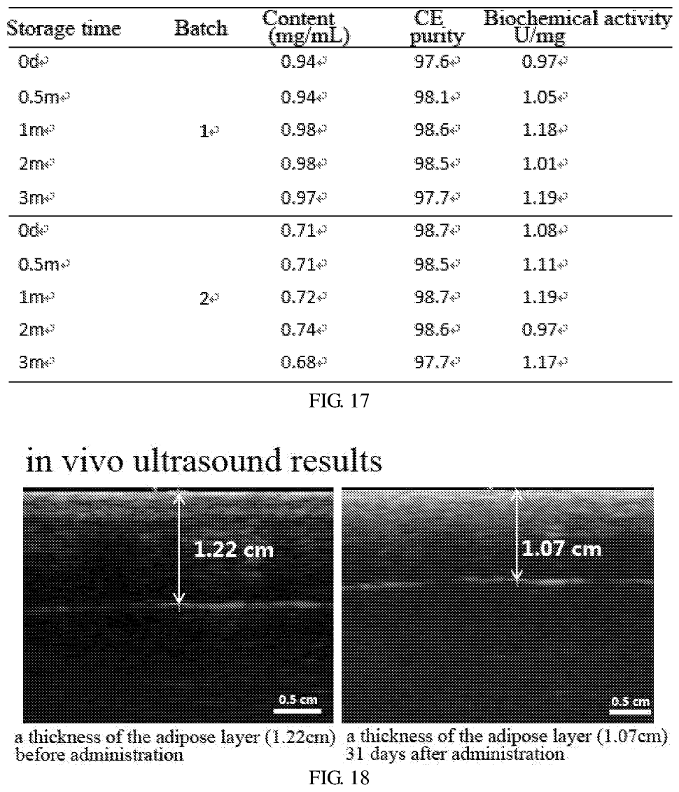

FIG. 17 shows that, freezing for three months at low temperature had no effects on biochemical activity of RJV001 drug substance.

### Embodiment 5, Lipolysis Experiments of RJV001 on Bama Minipig by Subcutaneous Injection (Pre-Study)

One application for the recombinant mutant collagenase of the present invention is lipolysis. The freeze-dried drug products were dissolved in saline and injected subcutaneously into the mini pigs; the blank control was injected with saline. The lipolysis effect was evaluated by ultrasound and anatomic observation of fat layer. The experimental scheme and results are as follows:

Objective: to study the pharmacodynamics of RJV001 in adipose tissue of Bama miniature pig model.

Preparation: RJV001 freeze-dried drug products

Preservation conditions: storage at 4-8° C. no more than 3 months

Purity: 98.6%

Animal model: Bama miniature pigs, female, about 70 kg, provided by Wujiang Tianyu Biotechnology Co., Ltd

Animal feeding environment: Bama miniature pigs were raised in an indoor pig house meeting AAALAC requirements. The room temperature was controlled at 16-26° C. and relative humidity was kept at 40-70%. The illumination controlled by fluorescent lamps lasted for 12 hours (8:00-20:00) with 12 hours in dark.

Animal-Feeding Food and Water Source: animals have unrestrained food and water supply. The corresponding equipments are provided by Beijing Keaoxieli Feed Co., Ltd. and verified. The water source is purified through a filtration system and meets human drinking standards by WHO. Water quality analysis is carried out twice a year, including heavy metals, nitrates, minerals, bacterial colonies and so on.

Experimental design and treatment process: three parts of adipose tissue in Bama miniature pigs were selected for study, namely left abdomen fat, right abdomen fat and back fat.

In the case of the left abdominal fat, each treatment site received a low dose (0.075 mg) of treatment. Six points of injection were given in each region with injection volume of 400 μL at each point and injection depth of 0.7 cm.

In the case of the right abdominal fat, each treatment site received a medium dose (0.15 mg) of treatment with six injection points in each region. Injection volume of each point was 400 μL and injection depth was 0.7 cm.

In the case of the back fat, each treatment site received a high dose (0.30 mg) of treatment with six injection points in each region. Injection volume of each point was 400 μL and injection depth was 0.7 cm.

The negative control group was injected at six points in two areas of the Bama miniature pig model.

Blood sample collection: 1 mL of blood was collected from Bama miniature pig at before and 0.5 hour or 1 hour after the treatment.

Experimental Observation and Result Evaluation:

(a) Ultrasound detection: every week after the first administration, thickness of subcutaneous adipose layer at the site of administration will be measured by ultrasound before injection. The same ultrasonic power is guaranteed for each ultrasonic test.

(b) Epidermal analysis: every other week skin surface of Bama miniature pigs is observed and photographed.

(c) Anatomy: all experimental animals will be dissected after euthanasia with pentobaibital sodium injection at the eighth week after administration, and each experimental site will be taken out and photographed.

(d) Pathology: each fat pad obtained from dissection is immersed in 10% formalin for at least 48 hours and sent to the tissue treatment laboratory. Inflammation is analyzed by H&E staining and tissue fibrosis is analyzed by Masson trichrome staining.

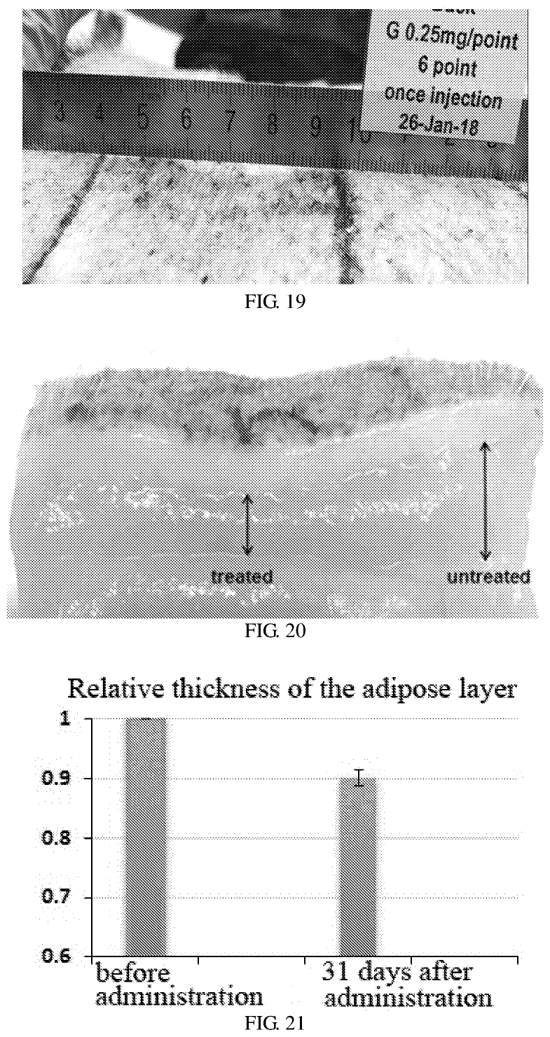

FIG. 18 shows changes of adipose layer thickness before administration and 31 days after administration. According to in vivo ultrasound results, the adipose layer thickness decreased from 1.22 cm before administration to 1.07 cm after 31 days of administration.

FIG. 19 shows local epidermal analysis at 31 days after single administration. It can be seen that there am obvious depressions on the local epidermis after administration, which indicates that the subcutaneous fat is effectively dissolved.

FIG. 20 shows physiological and anatomical results at 31 days after single administration. The anatomical results in FIG. 20 show that thickness of the fat layer in the administration area is significantly reduced when compared with that in the non-administration area, which is consistent with ultrasound results before the administration.

FIG. 21 shows that the relative adipose layer thickness of multiple sites decreased by 10% on average at 31 days after administration through in vivo ultrasound analysis.

Embodiment 6, Lipolysis Study of RJV001 on Bama Miniature Pig

One application for the recombinant mutant collagenase of the present invention is lipolysis. The freeze-dried drug products were dissolved in saline and injected into the mini pigs: the blank control was injected with saline. The lipolysis effect was evaluated by ultrasound and anatomic observation of fat layer. The experimental scheme and results are as follows:

Objective: to study pharmacodynamics of RJV001 in adipose tissue of Bama miniature pig model.

Preparation: RJV001, provided by Rejuven Dermaceutical Co., Ltd.

Reagent form: colorless liquid

Preservation Conditions: stable at 4-8° C. for 3 months

Purity: 98.7%

Batch: 20180602DSA

Animal model: Bama miniature pigs, 1 female, 2 male, about 30 kg, 6 months old, provided by Wujiang Tianyu Biotechnology Co., Ltd.

Animal feeding environment: Bama miniature pigs were raised in an indoor pig house meeting AAALAC requirements. The room temperature was controlled at 16-26° C. and relative humidity was kept at 40-70%. Illumination was controlled by fluorescent lamps and it lasted for 12 hours (8:00-20:00) with 12 hours in dark.

Animal-Feeding Food and Water Source: Animals have unrestrained food and water supply. The corresponding equipments are provided by Beijing Keao Xieli Feed Co., Ltd. and verified. The water source is purified through a filtration system and meets human drinking standards by WHO. Water quality analysis is carried out twice a year, including heavy metals, nitrates, minerals, bacterial colonies and so on.

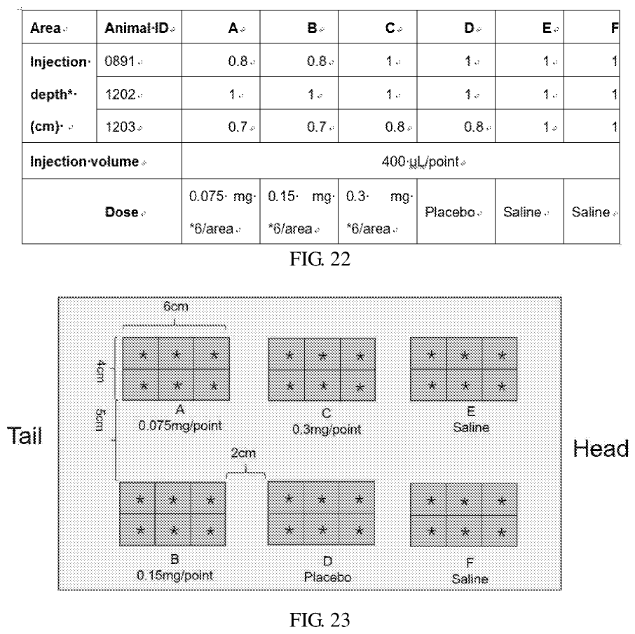

The experimental design and treatment process: 2 male and 1 female Bama miniature pigs were used to study the adipose tissue of their backs. Placement of the treatment area is shown in FIG. 22, and the treatment plan is shown in Table 1.

In the case of the back fat, each treatment site received different doses of treatment (0.075 mg. 0.15 mg. 0.30 mg and placebo) with six injection points in each region. Injection volume of each point was 400 μL. Before administration injection depths of different areas in different Bama miniature pigs were adjusted according to ultrasound results of adipose tissue to ensure that injection of RJV001 reached the basement membrane.

Placebo: sucrose 18.5 mg/mL, CaCl₂ 0.3 mg/mL, Tris 2.2 mg/mL.

Negative control group: inject normal saline and choose two areas of the Bama miniature pig model for six injection points with injection volume of 400 μL at each point.

Experimental Observation and Result Evaluation:

(a) Weight: record the weight of each animal every week.

(b) Ultrasound detection: every week after the first administration thickness of subcutaneous adipose layer at the site of administration will be measured by ultrasound before injection. The same ultrasonic power is guaranteed for each ultrasonic test. Such thickness is measured four times from different directions in each area.

(c) Epidermal analysis: every other week skin surface of Bama miniature pigs is observed and photographed.

(d) Anatomy: all experimental animals will be euthanized four weeks after administration. After Zoletil anesthesia appropriate amount of 10% KCl (i.v) is injected into the animals and the animals are killed by bloodletting. Take out each injection pad and take a picture of it. After euthanasia each experimental site is taken out and photographed.

(e) Pathology: each fat pad after dissection is immersed in 10% formalin for at least 48 hours and sent to the tissue treatment laboratory. Inflammation is analyzed by H&E staining and tissue fibrosis is analyzed by Masson trichrome staining. The parameters used by pathologists for evaluating and scoring are grades of 0 (normal), 1 (mild), 2 (moderate), 3 (moderate to severe or significant) and 4 (significant).

FIG. 22 shows the administration sites on the back of Bama miniature pigs.

FIG. 23 shows the changes of fat layer thickness before single administration and at 0-4 weeks after single administration. According to in vivo ultrasound results the fat layer thickness of low (A), medium (B) and high dose groups (C) was significantly different from that of placebo (D) and negative control areas (E. F) in the third and fourth week after administration.

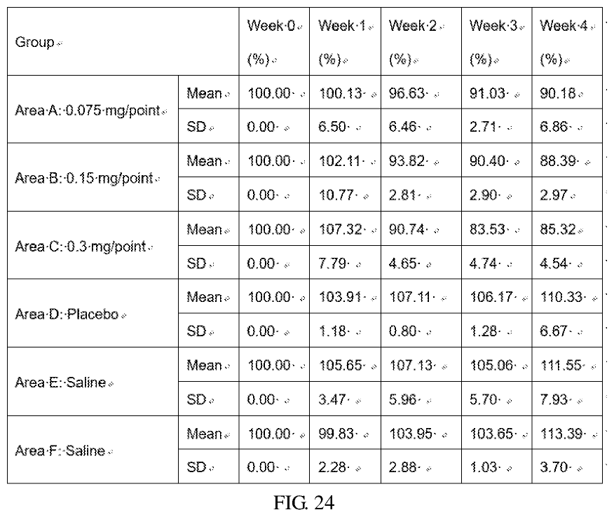

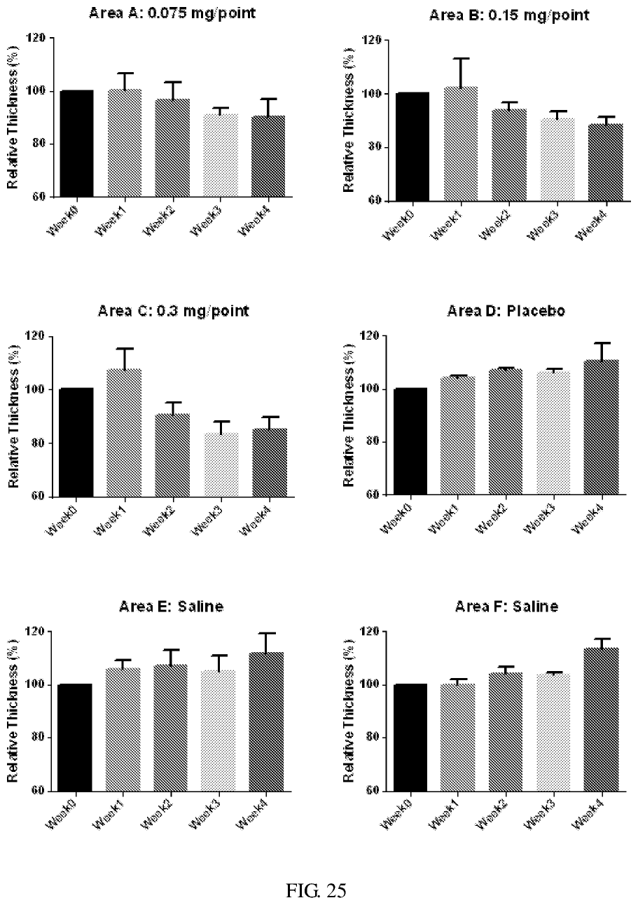

FIGS. 24-25 show the changes of adipose layer thickness before single administration and at 0-4 weeks after single administration.

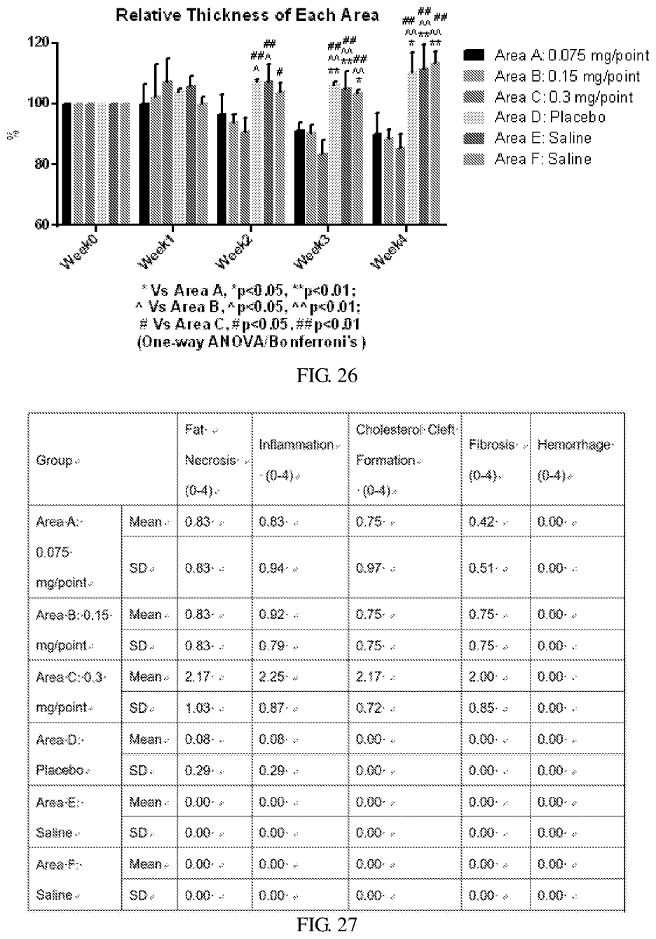

FIGS. 26-27 show the histopathological data, revealing an increasing trend in fat necrosis, inflammation, cholesterol fissures and fibrosis area for the high dose group (C).

Current experiments show that 0.075 mg dose can significantly reduce the thickness of adipose tissue in the 3rd and 4th weeks after administration. While by using 0.15 mg & 0.3 mg dosage a significant difference in the thickness of adipose tissue is observed from the 2nd week. In addition compared with saline injection, high doses (0.3 mg) result in significant fat necrosis, inflammation, cholesterol fissures and fibrosis.

FIG. 22 depicts the dosage regimen in lipolysis study of Bama miniature pig.

FIG. 23 depicts the treatment layout in lipolysis study of Bama miniature pig.

FIG. 24 depicts the mean relative thickness of each area in lipolysis study of Bama miniature pig.

FIG. 25 depicts the relative thickness of each area in lipolysis study of Bama miniature pig.

FIG. 26 depicts the relative thickness in lipolysis study of Bama miniature pig.

FIG. 27 depicts the histopathological statistics results in lipolysis study of Bama miniature pig.

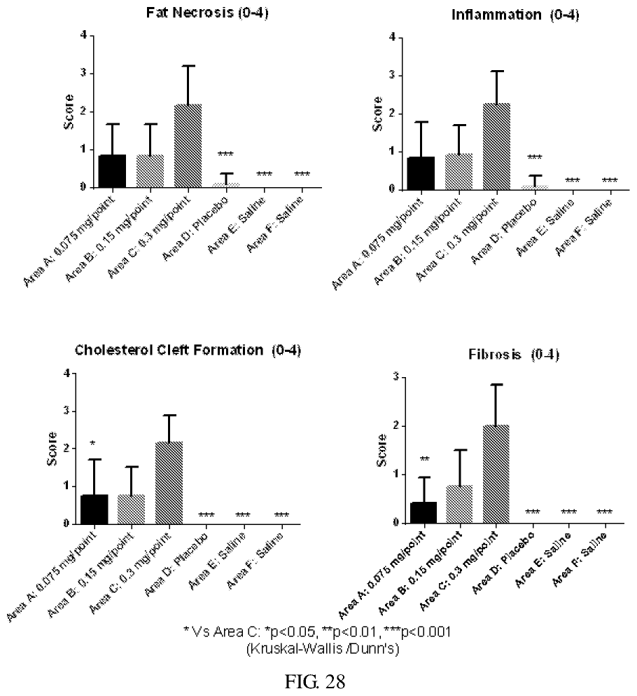

FIG. 28 depicts the histopathological results in lipolysis study of Bama miniature pig.

The preferred embodiments of the present invention are described in detail above, but the present invention is not limited thereto. Within the scope of the technical conception for the present invention, a variety of simple variants for the technical solution of the present invention can be made, including the combination of various technical features in any other suitable ways. These simple variants and combinations should also be regarded as the contents disclosed by the present invention and belong to the scope of protection of the present invention.

The present application claims the priority of the Chinese Patent Application No. 201810851432.1 filed on Jul. 13, 2018, which is incorporated herein by reference as part of the disclosure of the present application.

1. A composition comprising recombinant mutant collagenase with purity higher than 98%, wherein the recombinant mutant collagenase is Clostridium histolyticum collagenase H (ColH) with glutamic acid of 451 site mutated to aspartic acid, and the sequence of the recombinant mutant collagenase is shown as SEQ ID NO: 1.

2. A method for preparing recombinant mutant collagenase with purity higher than 98%, wherein the sequence of the recombinant mutant collagenase is shown as SEQ ID NO: 1 and the method comprises the following steps: (1) Constructing a strain expressing the recombinant mutant collagenase, wherein the recombinant mutant collagenase is Clostridium histolyticum collagenase H (ColH) with glutamic acid of 451 site mutated to aspartic acid; (2) Fermenting the strain expressing the recombinant mutant collagenase; (3) Capto Phenyl HS hydrophobic interaction chromatography: equilibrating the Capto Phenyl HS hydrophobic chromatography column, precipitating with ammonium sulfate and resuspending it, loading the supernatant to the Capto Phenyl HS hydrophobic chromatography column, washing and eluting the column, collecting an elution peak and obtaining first-collected solution; (4) Capto Q anion exchange chromatography: equilibrating the Capto Q anion exchange chromatography column, loading the solution collected in step (3) to the Capto Q anion exchange chromatography column, washing and eluting the column, collecting a main elution peak and obtaining solution collected for the second time; (5) Capto Octyl hydrophobic interaction chromatography: equilibrating the Capto Octyl hydrophobic interaction chromatography column, loading the solution collected in step (4) to the Capto Octyl hydrophobic interaction chromatography column, washing and eluting the column, collecting a main elution peak and obtaining solution collected for the third time; (6) Phenyl HP hydrophobic interaction chromatography: equilibrating the Phenyl HP hydrophobic interaction chromatography column, loading the solution collected in step (5) after high salt-concentration process to the Phenyl HP hydrophobic interaction chromatography column, washing and eluting the column, collecting a main elution peak and obtaining solution collected for the fourth time; (7) Source 15Q anion exchange chromatography: equilibrating the Source 15Q anion exchange chromatography column, loading the solution collected in step (6) to the Source 15Q anion exchange chromatography column, washing and eluting the column, collecting a main elution peak and obtaining solution collected for the fifth time; (8) Replacing the solution collected in step (7) with buffer through ultrafiltration, and obtaining a final product by concentrating, filtering, sterilizing and freeze-drying.

3. The method of claim 2, wherein the host strain used for expressing recombinant mutant collagenase in Step (1) is E. coli BL21 (DE3).

4. The method of claim 2, wherein the temperature of fermentation in Step (2) is from 27° C. to 32° C.

5. A composition comprising the recombinant mutant collagenase prepared by the method of claim 4.

6. The composition of claim 5, further comprising a pharmaceutically acceptable carrier.

7. The composition of claim 5, wherein a formulation of the composition is an injection or a topical agent.

8. The composition of claim 7, wherein the injection is a liquid injection or a powder injection, and the topical agent is a cream, an emulsion or a solution.

9. Use of the composition of claim 5 in the preparation of medicines, cosmetics or health products for reducing and/or removing fat, which is adipose tissue near skin surface, subcutaneous adipose tissue or lipoma.

10. Use of the composition of claim 5 in the preparation of medicines, cosmetics or health products for dissolving adipose tissue, reducing scar or losing weight.

11. A composition comprising recombinant mutant collagenase with purity higher than 98%, wherein the recombinant mutant collagenase is expressed in E. coli with glutamic acid of 451 site mutated to aspartic acid, and the sequence of the recombinant mutant collagenase is shown as SEQ ID NO: 1.

12. The composition of claim 1, further comprising a pharmaceutically acceptable carrier.

13. The composition of claim 1, wherein a formulation of the composition is an injection or a topical agent.

14. Use of the composition of claim 1 in the preparation of medicines, cosmetics or health products for reducing and/or removing fat, which is adipose tissue near skin surface, subcutaneous adipose tissue or lipoma.

15. Use of the composition of claim 1 in the preparation of medicines, cosmetics or health products for dissolving adipose tissue, reducing scar or losing weight.

16. A composition comprising the recombinant mutant collagenase prepared by the method of claim 2.

17. The composition of claim 16, further comprising a pharmaceutically acceptable carrier.

18. The composition of claim 16, wherein a formulation of the composition is an injection or a topical agent.

19. Use of the composition of claim 16 in the preparation of medicines, cosmetics or health products for reducing and/or removing fat, which is adipose tissue near skin surface, subcutaneous adipose tissue or lipoma.

20. Use of the composition of claim 16 in the preparation of medicines, cosmetics or health products for dissolving adipose tissue, reducing scar or losing weight.
